(19) United States
(12) Patent Application Publication
(10) Pub. No.: US 2026/0263548 A1
(43) Pub. Date: Sep. 10, 2026

(54) GASTROINTESTINAL HEALTH SUPPLEMENTS

(71) Applicant: BIMINI, LLC, Topeka, KS (US)

(72) Inventor: Samer AL-MURRANI, Topeka, KS (US)

(21) Appl. No.: 19/166,233

(22) PCT Filed: Mar. 18, 2024
(86) PCT No.: PCT/US2024/020401

§ 371 Date: Sep. 17, 2025

Related U.S. Application Data
(60) Provisional application No. 63/490,818, filed on Mar. 17, 2023.
(60) Provisional application No. 63/490,829, filed on Mar. 17, 2023.
(60) Provisional application No. 63/611,477, filed on Dec. 18, 2023.

(51) Int. Cl. A61K 36/81; A61K 9/00; A61K 9/16; A61K 35/00; A61K 35/57; A61K 35/60; A61K 35/612; A61K 35/744; A61K 35/747; A61K 36/185; A61K 36/42; A61K 36/48; A61K 36/76; A61K 36/9066; A61K 38/48

(52) U.S. Cl. CPC A61K 36/81; A61K 9/0056; A61K 9/1623; A61K 35/57; A61K 35/60; A61K 35/612; A61K 35/744; A61K 35/747; A61K 36/185; A61K 36/42; A61K 36/48; A61K 36/76; A61K 36/9066; A61K 38/4873; A61K 2035/115

(57) ABSTRACT

Multi-action dosage form health supplements for supporting animal health and well-being, in particular through supporting digestive health. The formulations comprise blends of active anti-inflammatory botanical/herbal agents and/or probiotics, preferably two or more species-specific probiotics and/or antibodies, such as IgY and/or IgG derived from eggs, colostrum, and/or serum. Methods of supporting digestive health.

### CROSS-REFERENCE TO RELATED APPLICATIONS

The present application claims the priority benefit of U.S. Provisional Patent Application Ser. No. 63/490,818, filed Mar. 17, 2023, entitled DIGESTIVE HEALTH SUPPLEMENTS, Ser. No. 63/490,829, filed Mar. 17, 2023, entitled SPECIES-SPECIFIC PROBIOTIC STRAINS AND ANTIBODY BLENDS, and Ser. No. 63/611,477, filed Dec. 18, 2023, entitled SPECIES-SPECIFIC PROBIOTIC STRAINS AND ANTIBODY BLENDS, each of which is incorporated by reference in its entirety herein.

### BACKGROUND OF THE INVENTION

### Field of the Invention

The present invention relates to formulated dosage form health supplements for supporting animal health and well-being, particularly gastrointestinal and/or digestive health benefits, which may extend to broader health benefits in the subject.

### Description of Related Art

Gut health and well-being is a common concern among pet owners. Many dogs and cats suffer from digestive issues from time to time or on a chronic basis. Humans also suffer from gastrointestinal and/or digestive health issues. There are many medications, supplements, special food diets, and the like which have been proposed to support gastrointestinal and/or digestive health. Yet, there remains a need for formulated dosage forms for supporting health and well-being through the gastrointestinal tract, which are palatable and easy to administer. Further, there remains a need for probiotics-based supplements that are more targeted for a particular species microbiome.

### SUMMARY OF THE INVENTION

The present invention is broadly concerned with formulated multi-action dosage form health supplements for supporting animal health and well-being. In particular, embodiments of the present invention concern supplements comprising ingredients that provide a beneficial digestive health effect on a subject, particularly on animals, such as pets, but can also impart the effect on humans. Regardless of whether used for people or pets, the formulated products may be advantageously produced with natural ingredients and/or using systems, processes, and facilities certified for the manufacture of human food. The products may have a variety of forms, including but not limited to, powders, chews, tablets, and liquids. The formulated health supplements may be particularly suitable as pet supplements for dogs and cats, as well as feed supplements for horses and other domesticated animals, as well as livestock. Additionally, or alternatively, the formulated health supplements may be suitable for human use.

Particular embodiments described herein concern blends of active anti-inflammatory botanical/herbal agents, such as Agrimony, Cramp Bark, Golden seal, Marshmallow (plant), Meadowsweet, Ashwagandha, Chasteberry, Ginkgo Biloba, Siberian Ginseng, Skullcap, St. John's Wort, Corydalis, Devil's Claw, Willow Bark, Burdock, Cleavers, Echinacea, Licorice, Turmeric, Nettle Leaf, Red Clover, as well as powdered extracts such as pumpkin seed powder, ginger root powder, chamomile flower powder, passionflower powder. Preferably, the formulations include at least one, at least two, at least three, at least four, and even more preferably at least five different anti-inflammatory botanical/herbal agents. Such embodiments may further include salmon oil, primrose oil, sunflower oil, and/or krill oil. Such embodiments may further include a proteolytic enzyme, such as Bromelain, Papain, or combinations thereof, as well as amylases or lipases. Such embodiments may further include Egg Powder. Such embodiments preferably further include a probiotics blend of two or more, preferably three or more of Enterococcus faecium, Lactobacillus acidophilus, Lactobacillus casei, Lactobacillus lactis, and Lactobacillus reuteri. These digestion-enhancing components are supported in an edible/ingestible base formulation, which can be a chew, powder, or liquid as exemplified herein.

Particular embodiments described herein also concern blends of species-specific probiotics, antibodies and beneficial molecules naturally occurring with antibodies, and other ingredients and components comprising health supplement blends that provide a beneficial in-location gastrointestinal health effect on a subject or remote health effects on other organ systems of the subject through metabolites absorbed through the elementary canal and transported throughout the body. The probiotics, antibodies, and other components may be encapsulated or unencapsulated.

The species-specific probiotic blends can advantageously be formulated to specifically benefit subjects of a particular species by including probiotic organisms derived from healthy hosts of the target species. The antibodies can be derived from avian species or mammalian species, can be used purified or unpurified, and may be targeted against specific viruses, bacteria, parasites, allergens or other molecules that cause gastrointestinal issues in a species-specific manner. The botanicals and oils can provide combinations of antioxidants, immune activity enhancing, anti-inflammatory and other beneficial molecules that together deliver a multi-modality mode of action in a species-specific manner.

### DETAILED DESCRIPTION

The present invention is concerned with formulations for animal and/or human gastrointestinal and/or digestive health, including general well-being through improved gut health and microbiome. As used herein, reference to an “animal” is intended to refer to non-human animals. In one or more embodiments, the products comprise an active formulation, which may include ingredients or mixtures of ingredients that provide improved or maintained digestive health for an animal, whether through an herbal or botanical action, natural enzymes, and/or through probiotics blends, as well as through tailored species-specific probiotics blends. The products may be suitable for oral administration by humans and/or to a variety of animals, including domestic pets (e.g., dogs, cats, etc.) and larger animals (e.g., horses) and livestock (e.g., cattle, sheep, etc.).

The products may be in the form of soft chews, baked goods, tablets, powders, gummies, oils, or liquids. Soft chews, baked goods, tablets, or gummies are typically provided as individual unit dosage forms, such that one or more chews, tablets, etc. can be administered to the animal to achieve the desired total dosage form for that day. Dosages can also be divided up such as by breaking or cutting the individual unit dosage forms into smaller pieces. Likewise, dosages can be administered more than one time per day to achieve the desired total dosage form for that day. Powder, oil, or liquid form can be provided as powder or liquid formulation, or can be in the form of capsules, compressed tablets, or the like. Liquid dosage forms can also be provided as dry product for reconstitution in water or other suitable vehicle before being administered to the animal. Powder, oil, or liquid can be packaged in bulk, e.g., in a container containing numerous dosages, such as a canister, bottle, jar, etc., along with one or more serving or dispensing devices, such as scoops, cups, or droppers of a size or sizes appropriate to measure out a desired unit dosage form of the formulation from the bulk amount. Such formulations may also be dispensed from the bulk amount via pre-measured pumps. Unit dosages (in any format) can also be provided as individual packets (e.g., packets containing a single dose of formulation).

As used herein, “unit dosage form” or “unit dosage” refer to physically discrete units suitable for administration to human or animal subject in need thereof. In some cases, unit dosage forms may be packaged individually. Each unit dosage contains a predetermined quantity of the active formulation in combination with any base formulation ingredients. Thus, individual soft chews, baked goods, tablets, or gummies are non-limiting examples of unit dosage forms, as well as any predetermined volume of the formulation as measured by a provided scoop, cup, dropper, or pre-measured pumps for portioning out a unit dosage form of a powder or liquid formulation from a bulk container. Unit dosage forms can also be administered in fractions or multiples thereof. In one or more embodiments, unit dosage forms can be packaged as a plurality of identical unit dosages packaged in a single container, which can be administered in segregated unit dosage forms. Non-limiting examples include containers containing numerous soft chews, baked goods, tablets, or gummies, or bottles or containers of bulk liquid or powder formulation.

### Active Formulations

The dosage forms include an active formulation, which refers to the components of the composition that are selected and included as having a beneficial impact on gastrointestinal and/or digestive health of the subject (i.e., those that play an active role in the gastrointestinal and/or digestive health of the subject, as compared to being included merely for taste, flavor, texture, etc.).

In one or more embodiments, a nutraceutical-based active formulation may comprise a variety of botanical and/or natural homeopathic ingredients or enzymes that may individually, or in combination, maintain or provide improved digestive health for an animal and/or human. In one or more embodiments, the active formulation comprises one or more ground, spray dried, freeze-dried or otherwise powdered active ingredients, herbal extracts, one or more enzymes, and/or one or more probiotics (or probiotic blends). In one or more embodiments, the active formulation comprises a blend of active ingredients, one or more powdered extracts, one or more enzymes, and one or more probiotics (or probiotic blends). In one or more embodiments, the blend of active ingredients comprises one or more botanical or herbal ingredients. In one or more embodiments, the blend of active ingredients comprises one or more of Agrimony, Cramp Bark, Egg Powder, Golden seal, Marshmallow (plant), Meadowsweet, Ashwagandha, Chasteberry, Ginkgo Biloba, Siberian Ginseng, Skullcap, St. John's Wort, Corydalis, Devil's Claw, Willow Bark, Burdock, Cleavers, Echinacea, Licorice, Nettle Leaf, Turmeric, and Red Clover. In one or more embodiments, the one or more powdered extracts comprises pumpkin seed powder, ginger root powder, chamomile flower powder, and/or passionflower powder. In one or more embodiments, the active ingredients are present in the active formulation at about 50% to about 90% (or about 60% to about 80%) by weight and/or the powdered extract(s) are present in the active formulation at about 5% to about 30% (or about 10% to about 20%) by weight. In one or more embodiments, the active formulation comprises about 200 pu to about 100,000 pu (or about 5,000 pu to about 20,000 pu) of papain. In one or more embodiments, the active formulation comprises about 1 gdu to about 100 gdu (or about 10 gdu to about 30 gdu) of bromelian. In one or more embodiments, the active formulation comprises about 0.1B cfu to about 10B cfu (or about 0.5B cfu to about 5B cfu) of the one or more probiotics (B=billion; cfu refers to “colony forming units”). In one or more embodiments, the one or more general probiotics comprises Enterococcus faecium, Lactobacillus acidophilus, Lactobacillus casei, Lactobacillus lactis, Lactobacillus reuteri, and/or blends thereof (including those derived from humans).

In one or more embodiments, the formulated product comprises an ingestible product comprising the active formulation of botanicals and probiotics, etc. incorporated into a base formulation, which may include a mixture of inactive or non-therapeutic ingredients.

In one or more embodiments, more advanced formulations are described herein, including those which are tailored to be species-specific. Such advanced formulations comprise probiotics and probiotic blends, antibodies and other molecules normally occurring with antibodies present in eggs and/or colostrum and/or serum from pre-immune or immunized (hyperimmune) animals from avian or mammalian species, and botanical and oil ingredients and associated animal and/or human health products comprising the probiotics or blends and antibodies. It will be appreciated that such hyperimmune globulin therapy can be used to treat or prevent against a variety of infectious diseases of viral or bacterial origin. A variety of organisms (donors, such as chickens) can be exposed or immunized against a pathogen to induce the production of specific antibodies or immunoglobulins against the antigenic agent used for immunization or exposure. Depending on the donor animal, the approach can be used to induce the production of specific immunoglobulins, including IgG, IgY, IgM, IgE, and/or IgA which can then be obtained from the donor organism or its eggs, blood, serum, or other biological products and used in the compositions. Such hyperimmune globulin therapy can also involve immunoglobulins derived from human donors or sources. This technique is commonly used to produce chicken eggs containing specific immunoglobulins of interest that have therapeutic effect against an antigenic agent when the egg components are included in an active formulation.

In certain embodiments, the advanced antibody formulations may be incorporated into products as dosage form pet supplements. In one or more embodiments, the products comprise an active formulation, which may include ingredients or mixtures of ingredients that provide improved or maintained gastrointestinal health or other health benefits for a target species. The advanced active formulation may comprise a variety of ingredients that may individually, or in combination, maintain or provide improved gastrointestinal health or other health benefits for a target species.

In one or more embodiments, the active formulation comprises a species-specific active ingredient or ingredients. The diverse microbial populations present in the gastrointestinal systems of different host species can differ dramatically due to different basal body temperatures, dietary habits, and immune systems. This in turn favors a unique microbial community that is specifically able to survive and proliferate optimally within the gastrointestinal system of the host species. The bacteria present in the microbiome are therefore host-optimized. This can provide a significant advantage when the species-specific probiotic is derived from a target species and used in a health supplement or product for the same target species. For example, species-specific probiotics derived from a dog host can provide an improved health benefit for a dog as compared to probiotics derived, for example, from humans.

In one or more embodiments, the species-specific active ingredient or ingredients comprises a species-specific probiotic or blend of species-specific probiotics. As used herein, the term “species-specific” means that the blend, supplement, or other health product includes at least one ingredient that has been shown to be particularly beneficial to a target species. Thus, the term “species-specific probiotic” refers to a probiotic organism that imparts a beneficial health effect on a particular species when ingested by a subject of that species, and in particular a species-specific probiotic is derived from one or more healthy host subjects of the same target species. Thus, the species-specific probiotic is derived from a microbiome of the same species of the animal to be treated. In one or more embodiments, the species-specific probiotic comprises a microorganism extracted or otherwise isolated from the gastrointestinal tract of a dog or cat. In other words, cat-specific probiotics can be extracted or isolated from a healthy donor cat and used to formulate cat-specific supplements. Likewise, dog-specific probiotics can be extracted or isolated from a healthy donor cat and used to formulate dog-specific supplements. It will be appreciated that this represents an important advancement in the state of the art, as many probiotics, even those formulated for animals, are microorganism strains isolated from human microbiomes.

In one or more embodiments, the species-specific active ingredient or ingredients comprises one or more probiotic selected from the group consisting of Enterococcus Faecium, Lacticaseibacillus Casei, Limosilactobacillus Fermentum, Limosilactobacillus Reuteri, Pediococcus Acidilactici, and mixtures thereof, and specifically a strain of the foregoing that has been derived or isolated from the same species as the target species. In one or more such embodiments, these probiotics may be used in a health product for dogs. Other canine-specific probiotics can include, but are not limited to, Lactobacillus reuteri, Lactobacillus acidophilus, Lactobacillus murinus, Bifidobacteria animalis, Lactobacillus animalis, Lactobacillus johnosonii, Lactobacillus sanfranciscensis, Lactobacillus paraplantarum, Lactobacillus fermentum, Lactobacillus rhamnosus, Lactobacillus salivarius, Bifidobacterium pseudolongum, and Levilactobacillus brevis.

In one or more embodiments, the species-specific active ingredient or ingredients comprises one or more probiotics selected from the group consisting of Lacticaseibacillus Paracasei, Limosilactobacillus reuteri, Pediococcus Acidilactici, and mixtures thereof. In one or more such embodiments, these probiotics may be used in a health product for cats. Other feline-specific probiotics include, but are not limited to, Lactobacillus acidophilus, L. salivarius, Lactobacillus johnsonii, L. reuteri, Lactobacillus sakei, L. plantarum “Victoria”, and L. rhamnosus.

Human-specific probiotics may include, but are not limited to, Bifidobacterium catenulatum, Bifidobacterium bifidum, Bacillus amyloliquefaciens, Bacillus licheniformis, Bacillus pumilus, Bacillus coagulans, Bifidobacterium breve, Bifidobacterium lactis, Bifidobacterium longum, Enterococcus faecium, Lactobacillus acidophilus, Lactobacillus brevis, Lactobacillus casei, Lactobacillus fermentum, Lactobacillus paracasei, Lactobacillus plantarum, Lactobacillus reuteri, Lactobacillus rhamnosus, Lactobacillus sakei, Lactococcus lactis, and Streptococcus thermophilus.

Exemplary probiotics include those sold by CanBioCin. It should be understood that other commercially available probiotics may also be used, as well as independently grown strains of probiotics, and custom-made blends. Other probiotic organisms not listed herein may also be included within the scope of the invention.

As described above, the species-specific probiotic or probiotic blends may be derived from one or more healthy host subjects of the target species. In one or more embodiments, the probiotics may be encapsulated, which can help protect against damage caused by low pH and gastrointestinal acids and enzymes. In one or more embodiments, the probiotics may be unencapsulated.

The species-specific probiotics and blends may have a number of advantageous characteristics. For example, the probiotics may be substantially free of antibiotic resistant genes and virulence genes, spore-forming or non-spore forming and can survive through the gastrointestinal tract of the target species/subject, are capable of adhering to the intestinal cell lining, may produce postbiotic metabolites that strengthen the animal subject epithelial barrier, and can exhibit immunological protection to the target through the regulation, stimulation, and modulation of immune responses.

Probiotic bacteria digest and ferment dietary fibers, which results in the production of a wide range of health-regulating compounds known as “postbiotic metabolites.” Producing these metabolites allows the probiotics to have effects far beyond where they are localized in the gut.

A more diverse microbiome promotes better health because different strains of probiotic bacteria produce different kinds of postbiotic metabolites. However, diversity alone is not enough; microbiome balance is also a critical factor that influences microbiome health. People who purchase probiotics frequently have the mistaken belief that more is better. It is now common to see products claiming to be superior because they contain 50 billion bacteria or even 100 billion bacteria per dose. The fact is, massive doses of just one or several strains of probiotic bacteria does not promote microbiome balance, they actually work against balance. Balance and greater diversity are imperative because these factors result in the production of a broader range of postbiotic metabolites. Some of the benefits of various types of postbiotic metabolites include reducing inflammation, regulating the acid/base balance in the GI tract, directly fighting pathogens, regulating digestion and absorption of nutrients, detoxification, regulating the immune system, gut-brain communication, and more.

Examples of some of the better known postbiotic metabolites include the following:
Essential nutrients: B-vitamins, vitamin K, and aromatic amino acids;
Short-chain fatty acids (SCFAs): acetic, propionic and butyric acid;
Glutathione;
Antimicrobial peptides (AMPs) classified as bacteriocins and defensins;
Phenyl lactic acid;
D-amino acids;
Hydrogen peroxide;
Volatile organic compounds (VOCs);
Phytoestrogens: equol, enterolactone, enterodiol;
Urolithin A and urolithin B; and
Fulvic acids

When probiotics are ingested the postbiotic metabolites they produce immediately begin to exert their health-promoting effects when they arrive in the small intestines. Here are some of the better-known health effects of postbiotic metabolites:
Anti-inflammatory activity;
Antimicrobial peptides kill pathogens (critical aspect of immune system);
SCFAs play important roles in regulating the immune system;
Weakly acidic compounds create the optimal intestinal acid/base balance;
Hydrogen peroxide suppresses the growth of fungi and yeasts like candida;
Fulvic acids bind and transport minerals into cells;
SCFA butyrate is the primary energy source for epithelial cell growth;
Glutathione has powerful antioxidant, detoxification & immune system enhancing effects;
Phytoestrogens equol/enterolactone/enterodiol bind to estrogen receptors: lower menopausal symptoms & risks to many diseases; and
Probiotic-produced nutrients (B-vitamins & amino acids): wide range of benefits associated with these nutrients.

Postbiotic metabolites are compounds that are master regulators of animal health. Postbiotic metabolites influence and regulate every organ system, including the brain and the immune system, However, pathological bacteria also produce postbiotic metabolites. The difference is that the postbiotic metabolites produced by probiotic bacteria have beneficial health-regulating effects, whereas the postbiotic metabolites produced by pathological bacteria have toxic effects. It is therefore, important to create and maintain a healthy microbiome balance. A healthy microbiome consists of predominantly beneficial probiotic bacteria. The postbiotic metabolites from strains of probiotic bacteria create a microbiome ecosystem that supports the growth and proliferation of probiotic bacteria and inhibits the growth of pathogens.

In one or more embodiments, the advanced active formulation further comprises a source of immunoglobulin G (IgG, IgY, etc.) and/or other nutrients. In one or more embodiments, the antibodies can be encapsulated to protect the antibodies against damage caused by the stomach acid, or the antibodies may be unencapsulated.

In one or more embodiments, the source comprises bovine colostrum, which as a whole can include a mixture of antibodies, anti-oxidants, and/or other nutrients (carbohydrates, proteins, vitamins, prebiotics, gastric enzyme inhibitors, etc.), as exemplified by “Colostrum First Milk Bovine” sold by Immuno-Dynamics. In one or more embodiments, the other nutrients comprise proteins, such as lactoferrin. The colostrum may be from cows, sheep, goats, or other large mammals such as buffalo or camels. In one or more embodiments, the colostrum is present in an amount of 10 mg to 5 grams, preferably 200 mg to 400 mg of colostrum per dosage form (e.g., 2-5 gram chew), depending on the species (e.g., cat vs dog). In one or more embodiments, the colostrum is present in an amount of from about 0.25% to about 75% by weight, based upon the total weight of the dosage form taken as 100% by weight, for example, from about 1% to about 65% by weight, for example, from about 3% to about 50% by weight, for example, from about 5% to about 50% by weight. For example, dosages provided in powder form (e.g., where a dosage form is a predetermined-sized scoop of powder), the amount of the various active ingredients can be varied widely without adversely impacting the form and format of the powder. However, other dosage forms, such as soft chews need a requisite amount of moisturizing agents and binders/carriers to hold the active ingredients, thus, providing a more limited range of inclusion of the active ingredients.

Colostrum is designed by nature to “protect” against pathogens, enhance the growth of beneficial bacteria, and augment the activity of the immune system when it is at its weakest. It contains two groups of proteins: Whey proteins which are soluble and caseins which are insoluble. Both components provide both nutritional and bioactive properties that complement the present invention. Casein contains peptides with opioid type activity that have been shown to decrease gastric emptying activity in rats. Casein is also thought to preserve the activity and aid in the absorption of other biologically active peptides by reducing their digestion by pancreatic enzymes by means of functioning as a competitive substrate. Additionally bovine trypsin inhibitor an enzyme that protects IgGs, growth factors and other bioactive proteins from proteolytic degradation is present at levels that are 100-fold higher in colostrum than in regular or mature milk. Therefore, casein possesses immune-regulatory, anti-bacterial and anti-inflammatory properties.

Whey proteins include immunoglobulins, lactoferrin, alpha-lactalbumin, beta-lactoglobulin, lactoperoxidase, glycomacropeptide, and several growth factors. Many of the Whey proteins possess biological activity that include activating immune signaling pathways, and reducing inflammation. Alpha-lactalbumin, for example, exhibits anti-microbial and anti-tumor activity in addition to binding calcium and zinc ions.

Colostrum also contains carbohydrates that include lactose, oligosaccharides, glycolipids, glycoproteins, and nucleotide sugars. Lactose for example, has many functions but can also act as a built-in prebiotic for colonic bacteria. Additionally, the glycosylated proteins present in colostrum also function as prebiotics due to the saccharide component being cleaved by bacterial glycosidases, thereby influencing the gut microbiome.

Other molecules in colostrum include fats and lipids and higher concentrations of vitamins and minerals, particularly vitamins, B2, B12, E and D and minerals such as calcium, copper, iron, zinc, magnesium, manganese, and phosphorus, some such as B vitamins act as postbiotics.

Immunoglobulins in colostrum are passively passed from mother to offspring. The major immunoglobulin in cow's colostrum is IgG. Bovine immunoglobulins naturally found in colostrum can help prevent pathogen binding to host cells, present pathogens to macrophages for destruction, stimulate the adaptive immune response, modify intestinal bacterial populations, and induce local immunoglobulin A production.

In other embodiments another source of specific IgG antibodies may be derived and concentrated or purified from the serum of hyper-immune cows. These antibodies can be combined with or replace the antibodies from colostrum as a source of IgG. Such serum derived antibodies can be available commercially from companies such as ImmunoLin (manufactured by Proliant Health Biologicals), but can also be generated by the immunization of cattle with specific antigens.

In addition to normal IgG, subjects can be induced to produce specific antibodies, including IgY, IgG, IgE, IgM, and/or IgA. For example, immunization of cows against human, dog, cat, or horse pathogens (hyperimmunization) results in the production of specific antibodies against said pathogens. The resulting hyperimmune colostrum can then help in preventing and treating infections in human, dogs, cats, horses or other species, respectively.

Other anti-microbial components of colostrum such as bioactive oligosaccharides may function as competitive inhibitors of toxigenic bacteria for binding sites on gut epithelial cells by mimicking epithelial cell surface carbohydrates. Other components of colostrum including Lactoperoxidase and Lysozyme are toxic to multiple gram-positive and gram-negative bacteria.

Notwithstanding all of the above colostrum also contains a plethora of cytokines, growth factors, insulin-like growth factors, epidermal growth factors, and more all of which are provided through colostrum naturally to help build immunity and enhance health.

In one or more embodiments, the source of immunoglobulins comprises egg, for example in an amount of 10 mg to 4 grams of egg powder, preferably 200 mg to 400 mg of egg powder per dosage form (e.g., 2-5 gram chew), depending on the species (e.g., cat vs dog). In one or more embodiments, the egg powder is present in an amount of from about 0.25% to about 75% by weight, based upon the total weight of the dosage form taken as 100% by weight, for example, from about 1% to about 65% by weight, for example, from about 3% to about 50% by weight, for example, from about 5% to about 50% by weight. In addition to providing a well-balanced food source, eggs contain a myriad of biologically active components. Naturally occurring egg proteins are distributed equally egg white and egg yolk, while lipids, vitamins and minerals are concentrated in egg yolk. Apolipoprotein B, apovitelloenin-1, vitellogenins, albumin, immunoglobulins (natural IgY), ovalbumin and ovotransferrin are the most abundant proteins in egg yolk. Egg white on the other hand is a gel-like structure that lacks lipids and is composed mainly of water but also contains structural proteins, glycoproteins, anti-bacterial proteins and peptides. Egg white is also characterized by the presence of four highly abundant protease inhibitors (ovostatin, ovomucoid, ovoinhibitor and custatin) of major digestive enzymes (pepsin, trypsin, and chymotrypsin) that may delay and protect against digestion of egg components, including any IgY antibodies, as they traverse the stomach.

In one or more embodiments, the advanced active formulation further comprises one or more sources of Immunoglobulin Y (IgY) and/or other nutrients. In one or more embodiments, the active formulation comprises immunoglobulin Y (IgY), such as IgY Max sold by IgY Nutrition. In one or more embodiments, the IgY may be derived from the eggs of avian species, such as chickens, ducks, geese, turkeys, and the like. Additionally, or alternatively, the IgY may be purified from a live avian species. In one or more embodiments, the active formulation comprises IgY antibodies from chickens hyperimmunized against of Clostridium perfringens α-toxin, Clostridium perfringens elongation factor Tu (EF-Tu), Clostridium perfringens necrotic enteritis B-like (NetB) toxin, Clostridium perfringens Pyruvate: Ferredoxin oxidoreductase (PFO), or Eimeria tenella elongation factor 1-α.

Exemplary sources of IgY and IgG antibodies include those sold by IgY Nutrition and Immuno-Dynamics respectively. It should be understood that other commercially available antibody sources may also be used, as well as independently developed IgY and IgG antibody sources. Other types of IgY and IgG antibody sources not listed herein may also be included within the scope of the invention. Preferably, when present, the advanced active formulation will include at least 2.5% by weight of total IgY and/or IgG antibody, more preferably, at least 3% by weight, even more preferably at least 5% by weight, preferably from about 20% to about 40% by weight, but in some cases up to 75% by weight, preferably from about 3% to about 65% by weight, based upon the total weight of the dosage form taken as 100% by weight. In one or more embodiments, the antibodies, whether avian or mammalian, can be derived from pre-immune or from specifically immunized (hyperimmune) animals against certain pathogens or allergens that disrupt the normal function of the gastrointestinal tract. It will be appreciated that a number of donor animals can be used to generate the suitable antibodies. Non-limiting examples include camelids (camels, llamas, alpacas, etc.) pigs, cows, goats, and sheep, dogs, cats, and the like, as well as avian sources, including emus, ostriches, poultry (turkeys, chickens, ducks, etc.). Depending on the host animal, the antibodies can be derived or obtained from the serum, colostrum, eggs, etc. of the donor animal.

In one or more embodiments, the antibodies can be purified or used unpurified, for example, by including yolk or whole egg powder or unmodified colostrum, which include additional advantages of containing host derived proteins and other factors. Egg powder (whole egg, egg yolk) can be prepared by breaking up raw whole eggs or egg yolks (as applicable) and mixing with water to create a slurry. The slurry is then frozen down, followed by grinding, spray drying, or otherwise processing into a fine powder. The egg powder can also be prepared by simply desiccating the eggs and then grinding them whole into a powder.

The antibodies, whether from pre-immune or immunized (hyperimmune) animals, function by targeting the specific pathogens, allergen, or toxins that they are raised against. For example, egg yolk, whole egg powders, or colostrum from pre-immune avian animals or mammals can offer some basal level of protection. However, immunizing the animals can boost the amount of specific antibodies against the antigenic agent used for immunization, giving even more protection to the target animal (dog, cat, human, etc.).

In one embodiment, the antibodies present in the active formulation (whether from eggs, colostrum, or serum) can come from animals that have been immunized against a variety of antigenic agents including, but not limited to viral, bacterial, fungal, or parasitic antigens. Non-limiting examples include: canine parvovirus, canine coronavirus, canine distemper virus, canine adenoviruses, canine parainfluenza, canine herpesvirus, feline immunodeficiency virus, feline coronavirus, feline parvovirus, feline calicivirus, feline herpesvirus, Staphylococcus sp.; Staphylococcus simulans; Streptococcus pyogenes (all subtypes); Escherichia coli; Klebsiella pneumoniae subsp. Pneumoniae; Shigella dysenteriae; Pseudomonas aeruginosa (Schroeter) Migula; Proteus hauseri; Streptococcus salivarius subsp. Salivarius; Streptococcus agalactiae; Streptococcus oralis; Streptococcus pneumoniae; Streptococcus sanguis; Streptococcus mutans; Cutibacterium acnes; Haemophilus influenzae; Salmonella enterica serovar Enteritidis; Salmonella enterica serovar Typhimurium; Salmonella enterica serovar Heidelberg, Cryptosporidium parvum, Clostridium sp. (e.g., Clostridium difficile), Toxoplasma gondii, and the like.

In one or more embodiments, the antibodies present in the active formulation (whether from eggs, colostrum or serum) can come from animals that have been immunized against a series of pathogenic viruses, bacteria, fungi, and/or parasites that may target production animal species but may from time to time jump into non-production animal species or to humans. Such zoonotic microorganisms remain important public health concerns.

In one or more embodiments, the advanced active formulation includes an antibody blend of immunoglobulins derived from eggs and immunoglobulins derived from colostrum and/or serum. In one or more embodiments, the antibody blend contains a blend of at least IgY and IgG. In one or more embodiments, the antibody blend comprises a blend of egg powder (whole egg or yolk) and first colostrum powder. In one or more embodiments, the blend comprises a weight ratio of egg powder to colostrum powder ranging from about 25:75 to about 75:25, for example from about 40:60 to about 60:40, and in some cases about 50:50.

In one or more embodiments, the advanced active formulation may further comprise one or more botanical ingredients (or blends), one or more powdered extracts, one or more enzymes, and/or one or more oils. Exemplary botanical ingredients include Agrimony, Cramp Bark, Egg Powder, Golden seal, Marshmallow (plant), Meadowsweet, Ashwagandha, Chasteberry, Ginkgo Biloba, Siberian Ginseng, Skullcap, St. John's Wort, Corydalis, Devil's Claw, Willow Bark, Burdock, Cleavers, Echinacea, Licorice, Nettle Leaf, and Red Clover. In one or more embodiments, the one or more powdered extracts comprise pumpkin seed powder, ginger root powder, chamomile flower powder, and/or passionflower powder. In one or more embodiments, the one or more oils comprise salmon oil, primrose oil, sunflower oil, and/or krill oil. In one or more embodiments, the active formulation may further comprise amino acids, amino sugar(s) (e.g., glucosamine), turmeric, sulfated glycosaminoglycans (e.g., chondroitin sulfate), and/or organosulfur compounds (e.g., methylsulfonylmethane).

In one or more embodiments, the species-specific active ingredient(s), source of antibodies or other nutrients, or other active ingredients may be encapsulated. A variety of encapsulation technologies suitable for antibodies and probiotics can be used, including but not limited to, Sol-gel technology or Metal-Organic Framework technology.

In one or more embodiments, the advanced active formulation comprises about 0.01B cfu to about 20B cfu (or about 0.1B cfu to about 12B cfu) of one or more species-specific probiotics (B=billion; cfu refers to “colony forming units”). In one or more embodiments, the active formulation comprises about 0.1% to about 75% (or about 10% to about 50%) by weight of the source of antibodies and/or other nutrients.

### Base Formulations

In one or more embodiments, the formulated product comprises an ingestible product comprising one or more of the active formulations described above (nutraceutical and/or advanced species-specific) incorporated into a base formulation, which may include a mixture of inactive or non-therapeutic ingredients. In one or more embodiments, the base formulation comprises one or more base ingredients selected from the group consisting of emulsifiers, antioxidants, humectants, thickeners, solvents, sweeteners (or other flavoring agents or palatants), texture modifiers, surfactants, anti-caking agents, carriers, binders, preservatives, and mixtures thereof. Additional base ingredients and additives may also be included without departing from the scope of the present invention.

In one or more embodiments, the base formulation comprises an emulsifier such as lecithin, carrageenan, guar gum, xanthan gum, and the like. In certain embodiments, the base formulation comprises glycerin. In one or more embodiments, the base formulation comprises a texture modifier, which may comprise oil(s), and the like. The oil(s) may include coconut oil, palm oil, vegetable margarine, or other oil with a high melting temperature, preferably those that are solid at room temperature. In one or more embodiments, the base formulation comprises a binder such as a flour. The flour may include garbanzo flour, pea flour, sweet potato flour, rice flour, peanut butter flour, or other vegetable, cereal, or grain flour derived from a natural source. In one or more embodiments, the base formulation comprises a preservative selected from the group consisting of sorbic acid, citric acid, vitamin E, Mixed Tocopherols, Rosemary Extract, and mixtures thereof. Various animal- or non-animal based flavoring agents, palatants, or other food/nutritional products may also be included in the base formulation, such as carob, catnip, chicken, chicken liver, chicken flavoring, flaxseed, bacon flavoring, duck flavoring, inulin, maltodextrin, and the like.

In one or more embodiments, the ingestible product comprises about 40% to about 80% by weight of the base formulation and/or about 20% to about 60% by weight of the active formulation. In one or more embodiments, the ingestible product comprises about 10% to about 20% by weight of glycerin. In one or more embodiments, the ingestible product comprises about 8% to about 14% by weight of the texture modifier. In one or more embodiments, the ingestible product comprises about 3% to about 5% by weight of the emulsifier. In one or more embodiments, the ingestible product comprises about 25% to about 50% by weight of the binder. In one or more embodiments, each dosage form of the ingestible product has a total weight of about 1 gram to about 20 grams (or about 2 grams to about 10 grams). However, the particular dose size (i.e., weight) of the ingestible product can be above or below these ranges and can be selected based on the corresponding weight of the subject.

Advantageously, the animal products described herein may be produced using systems, methods, and facilities that comply with 21 C.F.R. part 117 (cGMP Practice, Hazard Analysis, and Risk-Based Preventive Controls for Human Food). The products can therefore provide a high-quality dosage form pet and animal health supplement, which is particularly formulated to maintain or improve the digestive health of an animal. Similarly, the products can therefore provide a high-quality dosage form human health supplement, which is particularly formulated to maintain or improve the digestive health of a human subject.

### Methods

In use, the product comprising the active formulation and base formulation is administered to an animal or human. In one or more embodiments, the product may be orally administered. In one or more embodiments, the oral administration can occur via direct hand feeding at desired intervals, such as daily, twice daily, weekly, monthly, or as-needed. For example, the product can be administered to the animal or human in advance of a stressful situation that might lead to digestive health issues, or after noticing digestive health issues, after an illness, etc. In one or more embodiments, oral administration can also be carried out by placing the product in an area in which an animal will come into contact with (and ingest) the product. For example, in one or more embodiments, the product can be used as a top dressing or additive to the animal's feed. The formulations described herein may be used to support overall gastrointestinal health or to treat, ameliorate, improve, or aid in the recovery from particular gastrointestinal ailments, such as diarrhea, constipation, bloating, and/or gas.

In one or more embodiments, the dosage forms can be formulated into soft chews. The Soft Chew Treats are prepared by pre-mixing the ingredients in a mixer, and then passing through an extruder, preferably a cold extrusion process is used or cold pressing is used, in either case where the process is carried out at room temperature or slightly above (~24-27° C.). The process uses low temperatures (ambient or room temperatures) and pressures to nonetheless mix and combine the ingredients into a shaped chew, without damaging or inactivating the probiotics, antibodies, or other active ingredients. Notably, the Soft Chew formulations and the process of preparing the same do not require or include water, relying instead on humectants, such as the oils or glycerol, as described herein. Preferably, water is excluded from the formulation list and is not an added ingredient, except in very de minimis amounts (e.g., to pre-wet the extrusion equipment). More preferably, water is essentially absent from the formulation. As used herein “essentially absent” means that water is not an intentionally added ingredient, and further is not a separately added ingredient, it being appreciated that incidental amounts of water may be present in some of the ingredients used in the formulation. Thus, the resulting Soft Chews have low water activities of less than 0.75. As such, Soft Chews are shelf stable without the use of chemical preservatives, such as BHA (butylated hydroxyanisole) and BHT (butylated hydroxytoluene), which are preferably excluded and essentially absent from the Soft Chews. Preferably, the Soft Chews are shelf stable for at least 24 months. As used here, “shelf stable” means that the product remains stable under ambient conditions in an air sealed container without the need for refrigeration, i.e., the product does not spoil, rot, deteriorate, become stale, or rancid.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth formulations prepared in accordance with embodiments of the invention. It is to be understood, however, that these examples are provided by way of illustration, and nothing therein should be taken as a limitation upon the overall scope of the invention.

### Example I

Different base formulations were prepared according to Table 1 below. In each of the below base formulations, the ingredients were combined and mixed to form substantially homogenous mixtures.

TABLE 1
Base formulations.

| Base | Product | |
|---|---|---|
| Formulation | Type | Ingredients |
| 1 | Chew | Carob, Catnip, Chicken, Chicken Liver, |
| | | Coconut Oil, Garbanzo Flour, Glycerin, |
| | | Mixed Tocopherols, Palm Oil, Pea Flour, |
| | | Rosemary Extract, Sorbic Acid, |
| | | Sunflower Lecithin |
| 2 | Chew | Brewer's Yeast, Citric Acid, Flaxseed |
| | | Powder, Glycerin, Natural Bacon Flavor, |
| | | Natural Chicken Flavor, Palm Oil, |
| | | Sunflower Lecithin, Sweet Potato Flour |
| 3 | Chew | Citric Acid, Glycerin, Inulin, Natural |
| | | Duck Flavor, Palm Oil, Rice Flour, |
| | | Sunflower Lecithin |
| 4 | Powder | Citric Acid, Inulin, Natural |
| | | Chicken Flavor |
| 5 | Powder | Citric Acid, Maltodextrin, Natural |
| | | Chicken Flavor |
| 6 | Liquid | Glycerol, Water (minimal), |
| | | Chicken Flavor |

### Example II

An exemplary active formulation was prepared, which when combined with a base formulation (such as those described in EXAMPLE I), produces a product that maintains or improves digestive health when ingested by an animal or human. In particular, when an ingestible product containing an active formula produced according to Table 2 below is orally administered, the ingredients have been found to maintain or improve the digestive health of an animal or human.

TABLE 2
Active ingredients in 3.5-gram ingestible product.

| | |
|---|---|
| Ingredient | Amount |

| | | |
|---|---|---|
| Digestive Health Botanical Blend | 750 | mg |
| (Agrimony, Cramp Bark, Goldenseal, | | |
| Marshmallow and Meadowsweet)* | | |
| Ginger Root Powder | 50 | mg |
| Pumpkin Seed Powder | 50 | mg |
| Papain | 10,000 | pu |
| Bromelain | 20 | gdu |

| | |
|---|---|
| Probiotics Blend (Enterococcus faecium, | 1B cfu (at time |
| Lactobacillus acidophilus, Lactobacillus | of manufacture) |
| casei, Lactobacillus lactis, | |
| Lactobacillus reuteri)** | |

*Alternatively, the Digestive Health Botanical Blend may comprise Agrimony, Cramp Bark, Egg Powder, Golden seal, Marshmallow, and Meadowsweet.
**Alternative probiotics blend: Enterococcus faecium, Lactobacillus casei, Limosilactobacillus fermentum, Limosilactobacillus reuteri, Pedicoccus Acidilactic

### Example III

Exemplary powdered formulations were prepared comprising specifies-specific probiotic blends for dogs and cats. In particular, when a powdered product containing a species-specific probiotic blend is produced according to Table 3 (canine), a particularly beneficial health benefit is exhibited when ingested by dogs. Similarly, when a powdered product containing a species-specific probiotic blend is produced according to Table 4 (feline), a particularly beneficial health benefit is exhibited when ingested by cats.

TABLE 3
Canine specific probiotic formulation.

| | Amount (Ranges) |
|---|---|
| Active Ingredients | per Dosage Form |
| Enterococcus Faecium (Canine); | 0.1B cfu-12B cfu |
| Lacticaseibacillus Casei (Canine); | |
| Limosilactobacillus Fermentum (Canine); | |
| Limosilactobacillus Reuteri(Canine); | |
| Pediococcus Acidilactici (Canine)* | |
| OvaCol** | 0.25-75% by weight |

| Inactive Ingredients | Amount (Ranges) | |
|---|---|---|
| Peanut Butter Flour | 5-25% | by weight |
| Citric Acid | 0.1-10% | by weight |
| Maltodextrin | 10-90% | by weight |

*These strains are made by CanBioCin
**Blend of chicken egg powder (IgY Max ® (IgY Nutrition) or ActivEgg ® (Arkion Life Sciences)) and first milk colostrum from bovine (Immuno-Dynamics).

TABLE 4
Feline specific probiotic formulation.

| | Amount (Ranges) |
|---|---|
| Active Ingredients | per Dosage Form |
| Lacticaseibacillus Paracasei (Feline); | 0.1B cfu-12B cfu |
| Limosilactobacillus reuteri (Feline); | |
| Pediococcus Acidilactici (Feline) | |
| OvaCol* | 0.25-75% by weight |

| Inactive Ingredients | Amount (Ranges) | |
|---|---|---|
| Chicken | 1-50% | by weight |
| Chicken Liver | 1-50% | by weight |
| Citric Acid | 0.1-10% | by weight |
| Maltodextrin | 10-90% | by weight |

*Blend of chicken egg powder (IgY Max or ActivEgg ®) and first milk colostrum from bovine (Immuno-Dynamics).

### Example IV

Exemplary powdered formulations were prepared comprising species-specific probiotic blends for dogs and cats, with ingredients and amounts shown in each table below based on a per dosage form amount. In particular, when a powdered product containing a species-specific probiotic blend is produced according to Table 5, a particularly beneficial health benefit is exhibited when ingested by dogs with general gastrointestinal wellness issues. Similarly, when a powdered product containing a species-specific probiotic blend is produced according to Table 6, a particularly beneficial health benefit is exhibited when ingested by dogs having gastrointestinal and general mobility discomfort that could be associated with the normal again process or after rigorous activity sessions. Similarly, when a powdered product containing a species-specific probiotic blend is produced according to Table 7, a particularly beneficial health benefit is exhibited when ingested by dogs having gastrointestinal and general anxiety that could be associated with stressful situations such as separation anxiety or that associated with storms or fireworks. Similarly, when a powdered product containing a species-specific probiotic blend is produced according to Table 8, a particularly beneficial health benefit is exhibited when ingested by dogs having gastrointestinal and general skin and coat issues that could be associated with seasonal allergies.

TABLE 5
Canine specific probiotic formulation
for general gastrointestinal wellness.

| Actives | Inclusion (mg) | | % |
|---|---|---|---|
| Enterococcus Faecium (Canine); | 5B | cfu | 5 |
| Lacticaseibacillus Casei (Canine); | | | |
| Limosilactobacillus Fermentum (Canine); | | | |
| Limosilactobacillus Reuteri (Canine); | | | |
| Pediococcus Acidilactici (Canine) | | | |
| Folic Acid | 200 | mcg | 0.02 |
| Protease | 300 | PC | 0.075 |
| Vitamin B12 | 5 | mcg | 0.05 |
| Apple Pectin Powder | 10 | mg | 1 |
| Amylase | 200 | SKB | 0.2 |
| Cellulase | 20 | CU | 0.1 |

TABLE 6
Canine specific probiotic formulation for general
gastrointestinal wellness and mobility discomfort.

| Actives | Inclusion (mg) | | % |
|---|---|---|---|
| Enterococcus Faecium (Canine); | 5B | cfu | 5 |
| Lacticaseibacillus Casei (Canine); | | | |
| Limosilactobacillus Fermentum (Canine); | | | |
| Limosilactobacillus Reuteri (Canine); | | | |
| Pediococcus Acidilactici (Canine) | | | |
| Folic Acid | 200 | mcg | 0.02 |
| Protease | 300 | PC | 0.075 |
| Vitamin B12 | 5 | mcg | 0.05 |
| Apple Pectin Powder | 10 | mg | 1 |
| Amylase | 200 | SKB | 0.2 |
| Cellulase | 20 | CU | 0.1 |

| | | |
|---|---|---|
| Glucosamine Sulfate | 200 | 20 |
| Chondroitin Sulfate | 100 | 10 |

TABLE 7
Canine specific probiotic formulation for general
gastrointestinal wellness and anxiety.

| Actives | Inclusion (mg) | | % |
|---|---|---|---|
| Enterococcus Faecium (Canine); | 2B | cfu | 2 |
| Lacticaseibacillus Casei (Canine); | | | |
| Limosilactobacillus Fermentum (Canine); | | | |
| Limosilactobacillus Reuteri (Canine); | | | |
| Pediococcus Acidilactici (Canine) | | | |
| Folic Acid | 200 | mcg | 0.02 |
| Protease | 300 | PC | 0.075 |
| Vitamin B12 | 5 | mcg | 0.05 |
| Apple Pectin Powder | 10 | mg | 1 |
| Amylase | 200 | SKB | 0.2 |
| Cellulase | 20 | CU | 0.1 |

| | | |
|---|---|---|
| L-Theanine | 50 | 5 |
| L-Tryptophan | 50 | 5 |

TABLE 8
Canine specific probiotic formulation for general
gastrointestinal wellness and anxiety.

| Actives | Inclusion (mg) | | % |
|---|---|---|---|
| Enterococcus Faecium (Canine); | 5B | cfu | 2 |
| Lacticaseibacillus Casei (Canine); | | | |
| Limosilactobacillus Fermentum (Canine); | | | |
| Limosilactobacillus Reuteri (Canine); | | | |
| Pediococcus Acidilactici (Canine) | | | |
| Folic Acid | 200 | mcg | 0.02 |
| Protease | 300 | PC | 0.075 |
| Vitamin B12 | 5 | mcg | 0.05 |
| Apple Pectin Powder | 10 | mg | 1 |
| Amylase | 200 | SKB | 0.2 |
| Cellulase | 20 | CU | 0.1 |

| | | |
|---|---|---|
| L-Theanine | 50 | 5 |
| L-Tryptophan | 50 | 5 |

TABLE 9
Canine specific probiotic formulation for general
gastrointestinal wellness and skin and coat health.

| Actives | Inclusion (mg) | | % |
|---|---|---|---|
| Enterococcus Faecium (Canine); | 5B | cfu | 2 |
| Lacticaseibacillus Casei (Canine); | | | |
| Limosilactobacillus Fermentum (Canine); | | | |
| Limosilactobacillus Reuteri (Canine); | | | |
| Pediococcus Acidilactici (Canine) | | | |
| Folic Acid | 200 | mcg | 0.02 |
| Protease | 300 | PC | 0.075 |
| Vitamin B12 | 5 | mcg | 0.05 |
| Apple Pectin Powder | 10 | mg | 1 |
| Amylase | 200 | SKB | 0.2 |
| Cellulase | 20 | CU | 0.1 |

| | | |
|---|---|---|
| Salmon Oil | 50 | 5 |
| Sunflower Oil | 50 | 5 |

These active agent formulations can also be combined with one of the base formulations described in Example 1 above and used to create chews or top dressings.

1. A composition for supporting and maintaining a healthy digestive tract in a subject, the composition comprising a blend of an active agent formulation and a base formulation, wherein the active agent formulation comprises:
at least one, preferably two or more, anti-inflammatory botanical agent and/or oil, selected from the group consisting Agrimony, Cramp Bark, Golden seal, Marshmallow (plant), Meadowsweet, Ashwagandha, Chasteberry, Ginkgo Biloba, Siberian Ginseng, Skullcap, St. John's Wort, Corydalis, Devil's Claw, Willow Bark, Burdock, Cleavers, Echinacea, Licorice, Turmeric, Nettle Leaf, Red Clover, pumpkin seed powder, ginger root powder, chamomile flower powder, passionflower powder, salmon oil, primrose oil, sunflower oil, and/or krill oil;
at least one enzyme selected from the group consisting of proteolytic enzymes, amylases, and lipases; and
at least one probiotic or probiotic blend.

2. The composition of claim 1, wherein said probiotic or probiotic blend is a species-specific spore-forming or non-spore forming probiotic, specific for the species of the subject.

3. The composition of claim 1, wherein said active agent formulation further comprises chicken egg powder.

4. The composition of claim 1, wherein said probiotic or probiotic blend comprises one or more of Enterococcus faecium, Lactobacillus acidophilus, Lactobacillus casei, Lactobacillus lactis, and Lactobacillus reuteri.

5. The composition of claim 1, wherein said enzyme is Papain and/or Bromelain.

6. The composition of claim 1, wherein the active formulation comprises a blend of at least five different anti-inflammatory botanical agents and/or oils.

7. A composition for supporting and maintaining a healthy digestive tract in a subject, the composition comprising a blend of an active agent formulation and a base formulation, wherein the active agent formulation comprises:
one or more species-specific spore-forming or non-spore forming probiotic, specific for the species of the subject; and
one or more species-specific antibody from immunized or pre-immunized chicken egg powder, immunized or pre-immunized colostrum, and/or immunized or pre-immunized serum from a donor animal of the same species as the subject.

8. (canceled)

9. The composition of claim 7, wherein the active formulation comprises about 0.01 Billion colony forming units (cfu) to about 20 Billion cfu of the one or more species-specific spore-forming or non-spore forming probiotics selected from a group consisting of Bifidobacterium, Enterococcus, Lactobacillus, Bacillus, Pediococcus, Limosilactobacillus, Lacticaseibacillus, and Levilactobacillus.

10. The composition of claim 7, wherein the active formulation comprises from about 0.25 to about 75% by weight of a blend of immunized or pre-immune egg powder as a source of IgY antibodies and early milk colostrum as a source of IgG and other immune molecules.

11. The composition of claim 7, wherein the active formulation further comprises a source of fiber selected from one or more of the following: pumpkin seed powder, glucomannan, inulin, maltodextrin, pectin, oat bran, or rice bran.

12. The composition of claim 7, wherein the active formulation further comprises at least one, preferably two or more, anti-inflammatory botanical agent and/or oil, selected from the group consisting Agrimony, Cramp Bark, Golden seal, Marshmallow (plant), Meadowsweet, Ashwagandha, Chasteberry, Ginkgo Biloba, Siberian Ginseng, Skullcap, St. John's Wort, Corydalis, Devil's Claw, Willow Bark, Burdock, Cleavers, Echinacea, Licorice, Turmeric, Nettle Leaf, Red Clover, pumpkin seed powder, ginger root powder, chamomile flower powder, passionflower powder, salmon oil, primrose oil, sunflower oil, and/or krill oil.

13. The composition of claim 7, wherein the active formulation further comprises at least one enzyme selected from the group consisting of proteases, cellulases, amylases, lipases, and mixtures thereof.

14. A dosage form comprising or consisting of a composition according to claim 1, said dosage form being selected from the group consisting of powders, chews, tablets, gummies, oils, and liquids.

15. The dosage form of claim 14, wherein said dosage form is a soft chew, wherein said soft chew has a water activity level of less than 0.75.

16. The dosage form of claim 14, wherein said composition is essentially free of added water.

17. A method for supporting and maintaining a healthy digestive tract in a subject, the method comprising administering an effective amount of a composition according to claim 1 or a dosage form thereof to a subject in need thereof.

18. The method of claim 17, wherein said composition enhances or supports remote health effects in said subject beyond the gastrointestinal tract due to absorption of metabolites produced by the probiotics in the gastrointestinal tract of the subject.

19. The method of claim 17, wherein said administering step comprises placing said composition or dosage form in a location where the subject will come into contact with and ingest the composition or dosage form.

20. (canceled)

21. A composition for supporting and maintaining a healthy digestive tract in a subject, the composition comprising a blend of an active agent formulation and a base formulation, wherein the active agent formulation comprises one or more biologically active proteins selected from lactoferrin, lactoperoxidase, lysozyme, alpha-lactalbumin, beta-lactoglobulin, glycomacropeptide and ovotransferrin.

22. The composition of claim 21, wherein said active formulation comprises colostrum as a source of said lysozyme and/or egg powder as a source of said ovotransferrin.

23. The composition of claim 21, comprising two or more of said biologically active proteins.

24. The composition of claim 21, wherein the active formulation further comprises one or more species-specific spore-forming or non-spore forming probiotics selected from a group consisting of Bifidobacterium, Enterococcus, Lactobacillus, Bacillus, Saccharomyces, Pediococcus, Limosilactobacillus, Lacticaseibacillus, and Levilactobacillus.
